(19) United States
(12) Plant Patent Application Publication
(10) Pub. No.: US 2003/0126652 P1
(43) Pub. Date: Jul. 3, 2003

(54) Rose plant named 'TWOAGAIN'

(76) Inventor: Jerry F. Twomey, Leucadia, CA (US)

Correspondence Address: JERRY TWOMEY ROSES, 194 Avocado Street, Leucadia, CA, 92024, US

(21) Appl. No.: 10/035,286

(22) Filed: Jan. 3, 2002

(51) Int. Cl. A01H 5/00

(52) U.S. Cl. PLT/139

(57) ABSTRACT

A new rose cultivar of the Hybrid Tea class characterized by having very double, very long lasting, non-fading flowers with green outer petals and inner light pink petals with light sweet perfume.

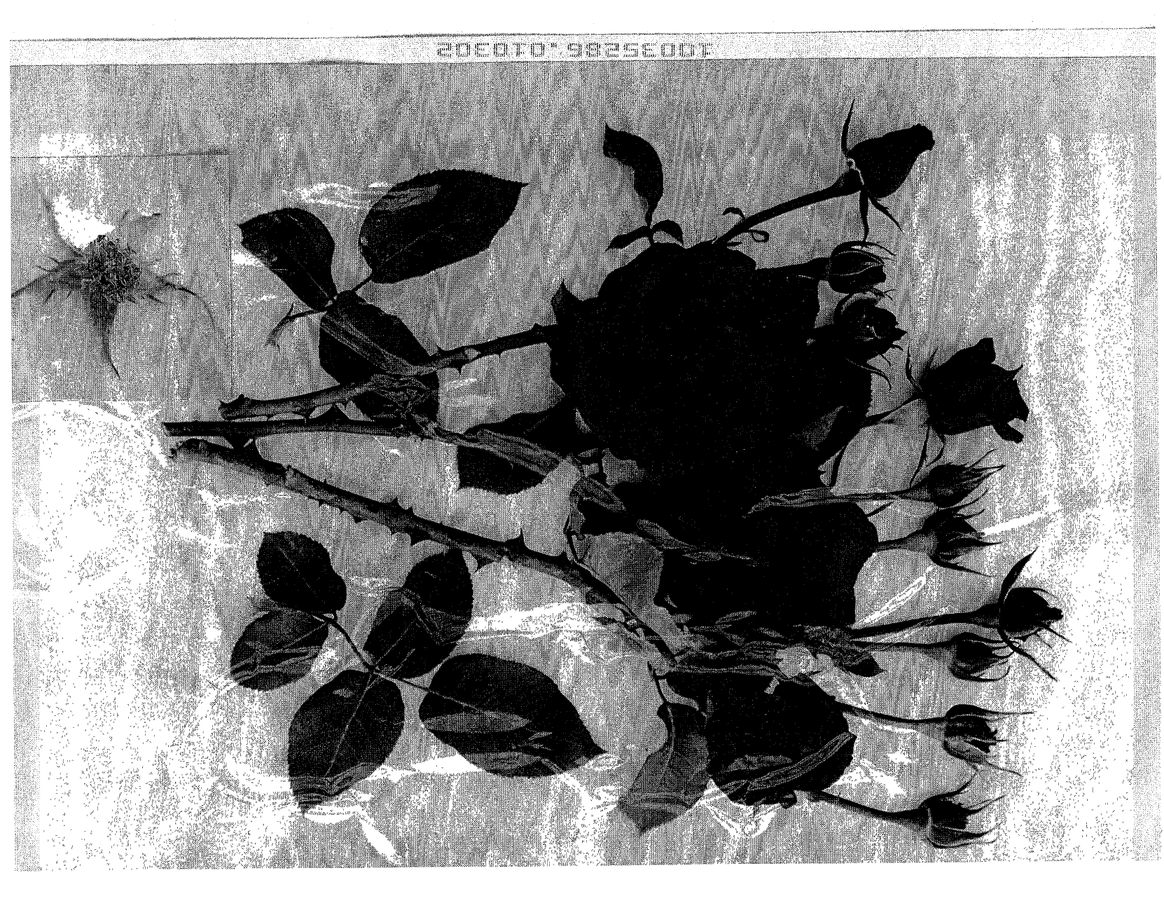

The generic name of this plant is designated as ‘TWOGREEN’. It is being marketed under the name of ‘Lady Joan’.

The present invention relates to a new and distinctive cultivar of rose plant of the Hybrid Tea class, which was originated by my crossing ‘Bahia’ as the seed parent with ‘Marina’ as the pollen parent.

Among the novel characteristics possessed by the new cultivar which distinguish it from:

(a) All other cultivars of which I am aware are:

(1) Green outer petals surrounding light pink inner petals.

(2) Does well as both a garden and greenhouse rose.

(3) Very long lasting as a cut flower.

(4) Consistent free bloomer.

(b) Seed parent: Bahia, Floribunda, orange blend.

Pollen parent: Marina, Floribunda, orange blend.

Asexual reproduction of the new rose cultivar by budding, as performed at Wasco. Calif., shows that the aforementioned and all other distinctive characteristics come true to form and are established and transmitted through succeeding propagations.

The accompanying drawing shows typical specimens of the vegetative growth and flowers of the new cultivar in different stages of development and as depicted in color as nearly true as it is reasonably possible to make the same in a color illustration of this character.

The following is a detailed description of my new cultivar, with color terminology in accordance with The Royal Horticultural Society Colour Chart (RHSCC). The terminology used in the color description herein refers to plate numbers in the aforementioned color chart, e.g. “53A” is plate “53A” of The Royal Horticultural Society Colour Chart.

Parentage:

Seed parent.—‘Bahia’, U.S. Plant Pat. No. 3525.

Pollen parent.—‘Marina’, U.S. Plant Pat. No. 3789.

Class: Hybrid Tea

The following observations are made of specimens grown outdoors in Monterey County, California, in the month of August, 2000.

### FLOWER

Blooming habit:

A. Bud:

(1) Size.—Sepals enclosed, 25-30 mm from base of sepals to top of bud.

(2) Form.—Ovoid.

(3) Sepals.—35 to 38 mm in length; 5 to 8 mm extension beyond bud; no serration shown on sepal edges. Generally 3 out of 5 sepals show appendages on each side, 5 to 10 mm length. Sepals show slight glandular development on edges. Heavy cotton matting on inner surface, fine matting on outer surface. Outside color 144A; inside 144C.

(4) Peduncle.—Length — 7 to 8 cm. Surface — primarily smooth, few small ½ to 1 mm prickles, few glandular hairs. Strength — firm. Color close — to 146B.

(5) Receptacle.—Shape round, 7-9 mm diameter, Color — 146C.

(6) Color.—When sepals first divide, outside bud petals near to 145C.

B. Bloom:

(1) Size.—When open — 10 to 11 cm.

(2) Borne.—about 2 to 3 per stem.

(3) Form.—Globular.

(4) Petalage.—Average — 42 to 46.

(5) Color.—Early open: outside and inside of outer petals 145C; inner petals near 38D, both surfaces; with base close to 36D. Aging: near to 36C.

C. Petals:

(1) Texture.—Firm.

(2) Appearance.—Flexed; satiny.

(3) Form.—Folding to pointed.

(4) Arrangement.—Regular.

(5) Petaloids in center.—Average 4 to 6.

(6) Persistence.—Holds petals well.

(7) Fragrance.—Light, sweet.

(8) Lasting quality.—On plant 14-16 days; cut 10-12 days.

### REPRODUCTIVE ORGANS

A. Stamen:

(1) Arrangement.—Regular around styles.

(2) Number.—Average 130-136.

(3) Length.—Average — 6 to 10 mm.

(4) Filaments.—Color — Close to 158D.

(5) Anthers.—Color — Close to 9D.

(6) Pollen.—Close to 16C.

B. Pistil:

(1) Styles.—Length: 15-20 mm exposed; number: 110 to 115; color: near to 50C; heavily coated with white hairs at base.

(2) Stigma.—Color — Close to 158C.

(3) Ovary.—Shape — oblong; 6-7 mm center cross section at sepal base.

C. Hips: Few hips but will set seed.

### PLANT

A. Form: Upright bush.

B. Growth: Garden: semi-vigorous, 1¼ m height; Greenhouse: vigorous, 2 m height.

C. Stem length: Basal break to flower bud tip: average — ½ m.

D. Foliage: Compound of 3 to 5 leaflets on bloom stem; 5 to 7 leaflets on main canes.

(1) Nature size.—5 leaflet — tip of leaflet to stipule base 16-18 cm.

(2) Quantity.—Stems well covered.

(3) Color.—New foliage upper side close to 137C; under side near 137D. Old foliage upper side near to 147A, under side near to 147B.

(4) Shape.—Oval with center pointed.

(5) Texture.—Firm, leathery, matte.

(6) Edge.—Serration on outer edge. Average 5 to 6 points per 1 cm, with up to 1 mm peak.

(7) Rachis.—Glandular hairs on lower side only. Few prickles on lower side: 3-4 mm length, hooked, 3-4 cm spacing.

(8) Stipules.—Length 15-18 mm; auricle to 4-5 mm. Edge of stipule to tip of auricle with many glandular hairs.

E. Resistance to disease: Powdery mildew, rust and blackspot: Good.

F. Wood:

New wood.—Color close to 146B, Bark — sooth.

Old wood.—Color close to 146A; Bark — smooth.

G. Thorns on mature wood:

Size.—4 to 8 mm. Thorns found primarily at lower half of stems.

Number per 10 cm.—Primary wood 5-6 thorns; Secondary wood 13-15.

Shape.—Thin. firm, generally straight outward from bark.

Color.—Early stage close to 164C; Aging close to 176A.

H. Winter hardiness: Average.

I claim:

1. A new and distinct rose cultivar of the hybrid tea class, substantially as herewith shown and described.
